(19) United States
(12) Patent Application Publication
(10) Pub. No.: US 2019/0033303 A1
(43) Pub. Date: Jan. 31, 2019

(54) SENSOR FOR DETECTION OF A TARGET SPECIES AND METHOD OF FORMING THE SAME

(71) Applicant: Cambridge Display Technology Limited, Godmanchester (GB)

(72) Inventors: SARAH TYLER, Godmanchester (GB); Jonathan Behrendt, Godmanchester (GB)

(73) Assignee: Cambridge Display Technology Limited, Godmanchester (GB)

(21) Appl. No.: 16/075,031

(22) PCT Filed: Feb. 3, 2017
(86) PCT No.: PCT/GB2017/050282

§ 371 Date: Aug. 2, 2018

(30) Foreign Application Priority Data
Feb. 3, 2016 (GB) 1601958.0

(51) Int. Cl. G01N 33/543; H01L 51/00; H01L 51/05; G01N 27/414

(52) U.S. Cl. CPC G01N 33/5438; G01N 27/4145; H01L 51/0545; H01L 51/0003

(57) ABSTRACT

A sensor for detection of a target species is provided. The sensor includes a capture layer on an organic semi-conductor to which biomolecules may be bound. The capture layer polymer is deposited from a non-aqueous solution and the polymer is insoluble in water and has reactive groups for interaction with the analyte directly or via a conjugated species. Organic electronic devices, for example organic thin-film transistors having the capture layer are also provided. Use of a capture layer provides a low cost high quality biosensor which may be reliably produced in large quantities.

### FIELD OF THE INVENTION

This invention relates to a sensor for detection of a target species, in particular a biosensor based on an organic electronic device particularly an organic thin-film transistor, and methods of forming said sensors.

### BACKGROUND OF THE INVENTION

Analysis of chemical and biological parameters of molecules and detection of chemical and biological species, for example proteins and DNA, is employed in a wide range of applications including drug discovery, medical diagnostics, protein and DNA analysis, detection and sequencing, forensic science and the like. Rapid analysis, a high degree of sensitivity, particularly at low concentrations of analyte and where samples may contain multiple components, is increasingly important. Where a sensor is to be used in a single-use application and is disposable, costs of production whilst retaining a desirable combination of technical characteristics is especially important.

Optical detection systems have been employed for some time in analysis and detection of chemicals and biomolecules and may be expensive. The use of organic electronic devices (OED) having an organic semi-conducting layer, for example organic thin-film transistors (OTFT), in sensors is known. Relatively low-cost, stable sensors have been developed which, upon interaction with a target species, provide a change in an electronic signal thereby to indicate qualitatively or quantitatively the presence of the target species.

Challenges remain however to ensure performance of the sensor is maintained during storage and in use, especially when used in aqueous media which may cause deterioration in electrical performance or reduced sensitivity, and there remains a need to reduce costs of production whilst maintaining high quality and the ability to produce the sensor in large scale quantities. Suitably the sensor may be tailored by incorporation of a biomolecule onto the surface of the sensor to enable binding to a particular target species allowing the sensor to be employed in a wide-range of different applications.

The performance of OEDs may be impaired in aqueous media and provision of one or more layers on the organic semi-conducting layer of the OED to ameliorate this problem is known.

Such a layer may however itself impair performance of the OED as it presents a physical barrier between the OED and the analyte species being detected. The layer must also be capable of interaction with the analyte species. In formation of the OED the organic semi-conductor layer may be deposited from a protic solvent, for example water and alcohols, or an aprotic polar solvent. The semi-conductor layer may be modified to allow attachment of a biomolecule but the procedure for bioattachment is typically carried out in an aqueous medium, for example a buffer which may render the organic semi-conductor film unstable or cause delamination.

WO2011/113935 describes a sensor comprising a transistor comprising a semiconductor layer and having a coupling/stabilisation layer covering at least part of the semiconductor layer. The coupling/stabilisation layer is deposited using plasma deposition to form the layer in situ. This process may provide a thin coupling/stabilisation layer but requires specialist apparatus to manufacture the sensor and is costly and complex. This is particularly disadvantageous given the demands on producing a low-cost disposable product.

WO 2013/082600 discloses a biosensor system including a detector configured to detect a change in an electrical property on a surface thereof having a passive layer disposed on a top surface of the detector, a hydrophobic layer disposed on the passive layer and a receptor-attachment material disposed on the hydrophobic layer and configured for binding to an analyte.

WO 2009/018496 discloses an array of microelectrodes, a first polymeric layer, a second polymeric layer and a capture molecule that is in physical communication with the second polymeric layer.

### SUMMARY OF THE INVENTION

The inventors have devised a sensor having a capture layer on an organic semi-conductor in which the aforementioned drawbacks with known sensors may be reduced or avoided. By providing a capture layer of polymer on an organic semi-conductor by deposition from a non-aqueous solution, where the polymer is insoluble in water and has reactive groups for interaction with the analyte directly or via a biomolecule bioconjugated with the capture layer, costly coating techniques may be avoided whilst providing a sensor having an organic semi-conductor layer which has not been materially compromised when applying the capture layer and a capture layer which is stable in aqueous media and which provides functionality for interaction with the species to be detected.

In a first aspect, the invention provides a method of making a layered structure for binding a biomolecule comprising:
forming an organic semi-conductor layer by a solution deposition method;
forming a capture layer on the organic semiconductor layer comprising the step of depositing a solution comprising a polar, non-aqueous solvent and a dissolved capture polymer onto the organic semi-conductor layer wherein the capture polymer comprises moieties adapted to bind to a biomolecule.

In a second aspect the invention provides a sensor or a precursor thereof for detection of a target species comprising an organic electronic device which comprises a layered structure for binding to a biomolecule capable of interacting with the target species to be detected, the layered structure comprising an organic semi-conducting layer and a capture layer carried on the semi-conducting layer, the capture layer comprising a polymer which is substantially insoluble in water and having a sample surface comprising moieties adapted to bind to the said biomolecule thereby to enable detection of the target species in use.

The capture layer is carried on the semi-conducting layer and is suitably deposited on and in contact with the semi-conducting layer.

The capture layer suitably covers at least part and preferably all the organic semi-conductor layer. Suitably, the capture layer protects underlying layer especially when aqueous media are used as a vehicle for the target species. The capture layer further provides a sample surface having a functional capability comprising reactive moieties to which a biomolecule may be bound to allow detection of target species.

In another aspect, the invention provides an organic electronic device for use in a sensor according to the second aspect of the invention. The OED is suitable for use as a sensor which comprises a layered structure for binding to a biomolecule, the layered structure comprising an organic semi-conducting layer and a capture layer carried on the semi-conducting layer, the capture layer comprising or consisting of a capture polymer which is substantially insoluble in water and having a sample surface comprising moieties adapted to bind to the said biomolecule thereby to enable detection of a target species in use.

The capture layer polymer has a structure that renders it soluble in polar solvents but insoluble in water. The biological compounds to be bound to the sample surface of the capture layer are suitably deposited from water or aqueous solution. The sample surface of the capture layer and preferably the capture layer is suitably stable in contact with water or an aqueous solution to allow the biological compound to be bound to the surface.

The capture layer may also act as a barrier layer. Water may undesirably degrade performance of an OTFT and the capture layer desirably reduces the risk of such degradation.

We have found that by careful selection of the polymer component of the capture layer and a suitable solvent for the polymer, the polymer may be deposited from a polar non-aqueous solvent using known deposition techniques to provide a high quality, stable, low cost sensor. This allows the capture layer to be formed without causing damage or delamination to the underlying organic semi-conducting layer. A sensor having an organic semi-conductor layer which is not impaired and a capture layer adapted for modification by binding a biomolecule and which is obtainable by a conventional solution deposition method may be obtained without the cost and complexity associated with using specialist techniques such as plasma deposition.

Suitably, the organic electronic device is an organic thin film transistor comprising a source electrode and a drain electrode defining a channel region therebetween. A semi-conducting layer suitably extends across the channel region and is in electrical contact with the source and drain electrodes. The OTFT further suitably comprises a gate electrode; and a gate dielectric between the gate electrode and the semi-conducting layer and the source and drain electrodes.

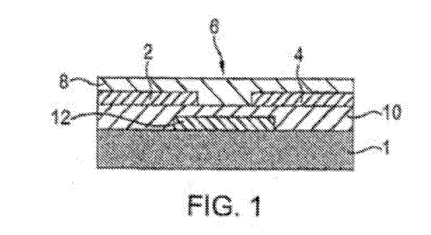

In other embodiments, The organic thin film transistor may be a bottom gate thin film transistor as illustrated in FIG. 1 in which the gate is provided on a bottom side of the device to form a so-called bottom-gate organic thin film transistor. An example of such a bottom-gate organic thin film transistor is shown in FIG. 1. The bottom-gate structure illustrated in FIG. 1 comprises a gate electrode 12 deposited on a substrate 1 with an insulating layer 10 of dielectric material deposited thereover. Source and drain electrodes 2, 4 are deposited over the insulating layer 10 of dielectric material. The source and drain electrodes 2, 4 are spaced apart with a channel region 6 located therebetween over the gate electrode. An organic semiconductor 8 is deposited in the channel region 6 and may extend over at least a portion of the source and drain electrodes 2, 4.

In a preferred embodiment, the OTFT comprises a bottom gate with a bottom contact or a bottom gate, top contact arrangement. Examples of suitable OTFT structures known in the art and suitable for use in the present invention are described in “Organic Thin Film Transistor Integration” by Li, Nathan, Wu and Ong—[2011] Wiley-VCH ISBN 978-3-527-40959-4.

An organic field effect transistor (OFET) may be employed, for example an electrolytically gated transistor where the top gate is provided by the analyte solution. An example of an OFET is provided in Organic Electronics January 2012; 13(1):1-6. DOI: 10.1016/j.orgel.2011.09.025. The paper published in Adv. Mater. 2014, 26, 1319-1335 sets out OFETs which may be employed in the present invention.

In one embodiment, the invention provides a flexible organic electronic device which comprises an organic field effect transistor and a layered structure for binding to a biomolecule, the layered structure comprising an organic semi-conducting layer and a capture layer carried on the semi-conducting layer, the capture layer comprising a polymer which is substantially insoluble in water and having a sample surface comprising moieties adapted to bind to the said biomolecule thereby to enable detection of a target species in use.

The OSC layer may comprise or consist of one or more organic semiconducting materials. Suitably the OSC layer comprises a polymer, which may be a homopolymer or a copolymer comprising two or more different repeat units, and / or may comprise a non-polymeric (small molecule) semi-conductor. Polymers can provide excellent film-forming capabilities and advantageously may be employed in combination with one or more small molecule organic semiconductors.

The polymer may be a non-conducting polymer material or a semiconducting polymer material. The polymer suitably is selected to overcome the low solubility and poor film forming properties which may arise with some organic semiconducting small molecules, e.g.

those known to the skilled person as described in the prior art such as Smith et. al., Applied Physics Letters, Vol 93, 253301 (2008); Russell et. al., Applied Physics Letters, Vol 87, 222109 (2005); Ohe et. al., Applied Physics Letters, Vol 93, 053303 (2008); Madec et. al., Journal of Surface Science & Nanotechnology, Vol 7, 455-458 (2009); and Kang et. al., J. Am. Chem. Soc., Vol 130, 12273-75 (2008).

Suitable polymers for use in the OSC layer include polymers comprising one or more repeat units selected from fluorene, triarylamine and benzothiophene .

If the polymer isa semiconducting polymer, the polymer is preferably a conjugated polymer comprising a repeat unit of the following formula (I):

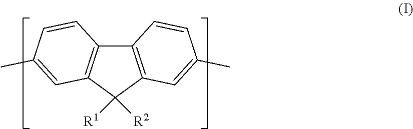

wherein R¹ and R² are the same or different and each is selected from the group consisting of i) hydrogen, ii) an alkyl group having from 1 to 16, preferably 1 to 12 and more preferably 4 to 12 carbon atoms, iii) an aryl group having from 5 to 14 carbon atoms, and iv) a 5- to 7-membered heteroaryl group containing from 1 to 3 sulfur atoms, oxygen atoms and/or nitrogen atoms, said aryl group or heteroaryl group being unsubstituted or substituted with one or more substituents selected from an alkyl group having from 1 to 16 carbon atoms and an alkoxy group having from 1 to 16 carbon atoms.

An aryl group R¹ or R² is preferably substituted or unsubstituted phenyl with one or more substituents selected from an alkyl group having from 1 to 12, preferably 4 to 8, carbon atoms and an alkoxy group having from 1 to 12, preferably 4 to 8, carbon atoms

In a preferred embodiment, the semiconducting polymer material is a conjugated polymer comprising the repeat unit (I), wherein R¹ and R² are the same or different and each is selected from the group consisting of an alkyl group having from 4 to 12 carbon atoms and a phenyl group, said phenyl group being unsubstituted or substituted with one or more substituents selected from an alkyl group having from 4 to 8 carbon atoms and an alkoxy group having from 4 to 8 carbon atoms.

The semiconducting polymer is suitably a conjugated polymer and may comprise the repeat unit (I) and further comprise a repeat unit of formula (II):

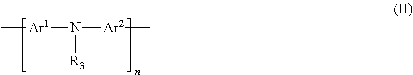

wherein Ar¹ and Ar² are the same or different and each is selected from an aryl group having from 5 to 14 carbon atoms and a 5- to 7-membered heteroaryl group containing from 1 to 3 sulfur atoms, oxygen atoms and/or nitrogen atoms, said aryl group or heteroaryl group being unsubstituted or substituted with one or more substituents selected from an alkyl group having from 1 to 16 carbon atoms and an alkoxy group having from 1 to 16 carbon atoms; R³ is an alkyl group having from 1 to 8 carbon atoms or a phenyl group which may be unsubstituted or substituted with one or more alkyl groups having from 1 to 8 carbon atoms; and n is an integer greater than or equal to 1, preferably 1 or 2.

Preferably Ar¹ and Ar² are each a phenyl group and R³ is an alkyl group having from 1 to 8 carbon atoms or a phenyl group which may optionally be substituted with one or more alkyl groups having from 1 to 8 carbon atoms, for example TFB poly-[9,9′-dioctylfluorene-co-N-(4-butylphenyl)-diphenylamine].

Examples of preferred polymers include those comprising repeat units of one or more of the following formulae:

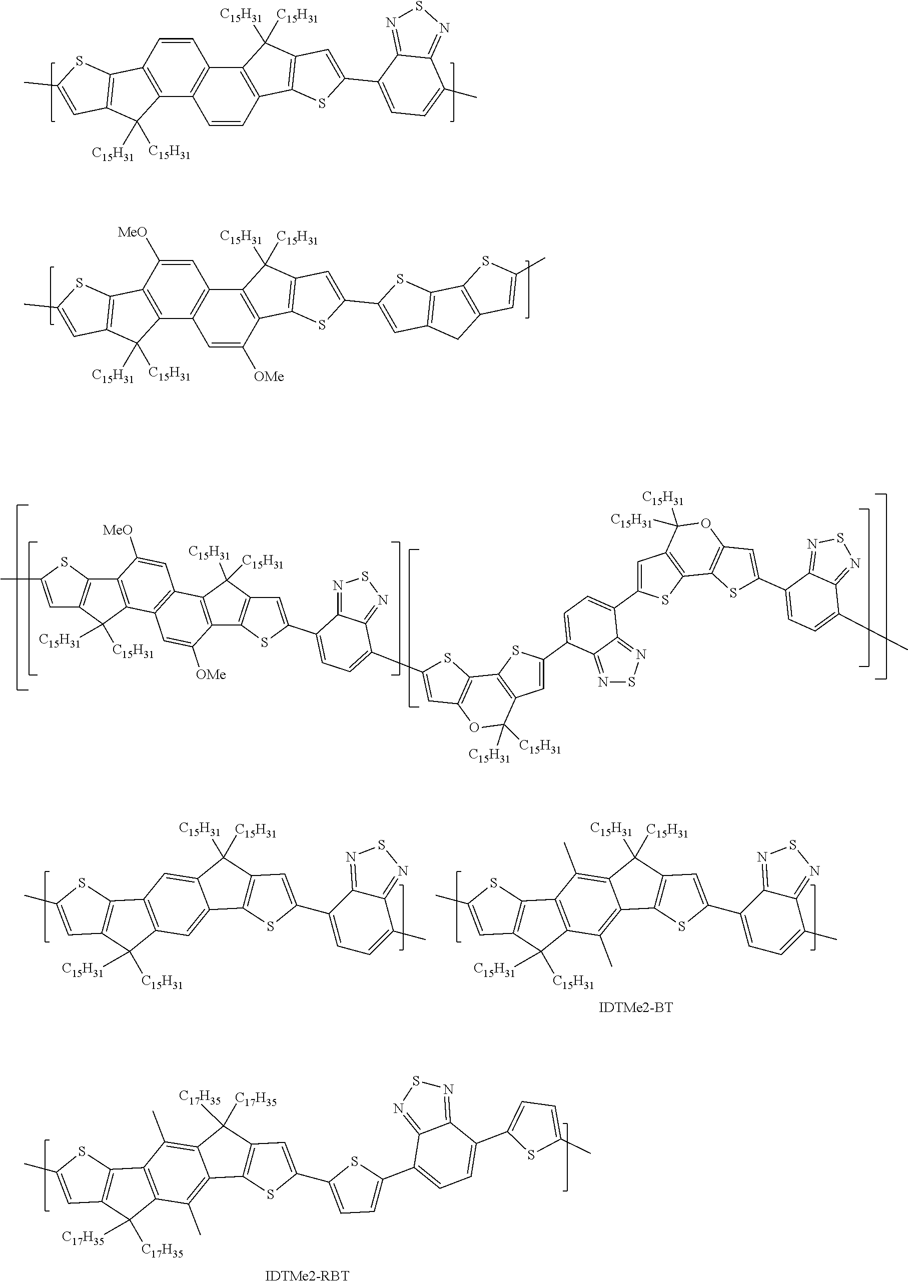

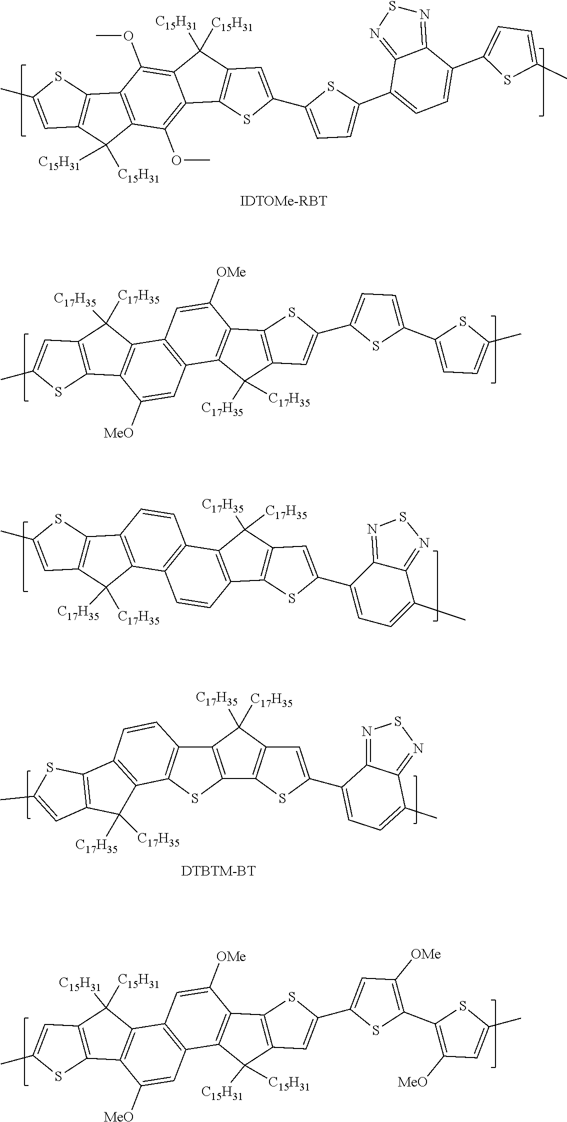

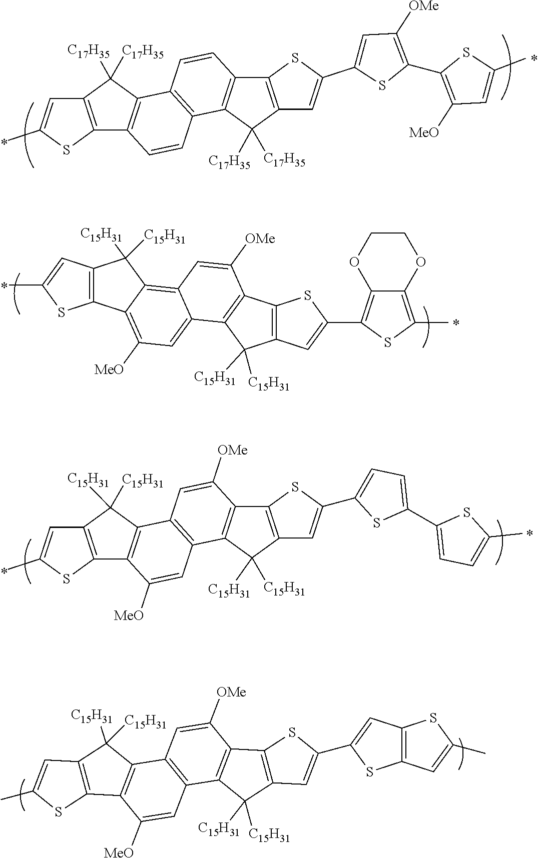

Examples of blends of semi-conductors (semiconductor-semiconductor or semiconductor—insulator) in the literature include Smith et. al., Applied Physics Letters, Vol 93, 253301 (2008); Russell et. al., Applied Physics Letters, Vol 87, 222109 (2005); Ohe et. al., Applied Physics Letters, Vol 93, 053303 (2008); Madec et. al., Journal of Surface Science & Nanotechnology, Vol 7, 455-458 (2009); and Kang et. al., J. Am. Chem. Soc., Vol 130, 12273-75 (2008). WO 2005/055248 discloses the preparation of organic thin film transistors in which the semiconductor layer is a blend of a pentacene derivative with a semiconductor binder such as a poly(triarylamine) or poly(9-vinylcarbazole) deposited from a solution thereof in a solvent.

Any known small molecule organic semiconductor may be employed. The small molecule OSCs may be a substituted pentacene or a fused thiophene. Examples of suitable substituted pentacenes and organic semiconducting compounds include compounds of formula (III)

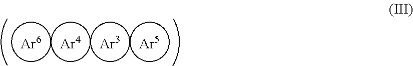

wherein Ar³, Ar⁴, Ar⁵ and Ar⁶ independently comprise monocyclic aromatic rings and at least one of Ar³, Ar⁴, Ar⁵ and Ar⁶ is substituted with at least one substituent X, which in each occurrence may be the same or different and is selected from the group consisting of (i) unsubstituted or substituted straight, branched or cyclic alkyl groups having from 1 to 20 carbon atoms, alkoxy groups having from 1 to 12 carbon atoms, amino groups that may be unsubstituted or substituted with one or two alkyl groups having from 1 to 8 carbon atoms, each of which may be the same or different, amido groups, silyl groups and alkenyl groups having from 2 to 12 carbon atoms, or (ii) a polymerisable or reactive group selected from the group consisting of halogens, boronic acids, diboronic acids and esters of boronic acids and diboronic acids, alkylene groups having from 2 to 12 carbon atoms and stannyl groups, and wherein Ar³, Ar⁴, Ar⁵ and Ar⁶ may each optionally be fused to one or more further monocyclic aromatic rings, and wherein at least one of Ar³, Ar⁴, Ar⁵ and Ar⁶ comprises a 5- to 7-membered heteroaryl group containing from 1 to 3 sulfur atoms, oxygen atoms, selenium atoms and/or nitrogen atoms.

One or more of Ar⁵ and Ar⁶ may be fused to a further aryl group Ar⁷ or Ar⁸ respectively wherein Ar⁷ and Ar⁸ independently represent a monocyclic aromatic ring unsubstituted or substituted with one or more substituents X, wherein the said monocyclic aromatic ring is preferably a 5- to 7-membered heteroaryl group containing from 1 to 3 sulfur atoms, oxygen atoms, selenium atoms and/or nitrogen atoms. Where present, Ar⁷ or Ar⁸ may be fused to a further aryl group Ar⁹ or Ar¹⁰ respectively wherein Ar⁹ or Ar¹⁰ independently represent a monocyclic aromatic ring unsubstituted or substituted with one or more substituents X, wherein the said monocyclic aromatic ring is preferably a 5- to 7-membered heteroaryl group containing from 1 to 3 sulfur atoms, oxygen atoms, selenium atoms and/or nitrogen atoms.

An especially preferred small molecule is a compound of structure:

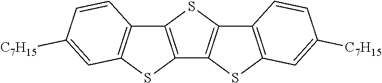

Other examples of suitable polymers and small molecule semiconductors are described in U.S. Pat. No. 9,159,926, the contents of which are incorporated herein by direct cross-reference.

The invention enables low-cost OEDs, for example OTFTs, to be used to produce a sensor without the need for recourse to expensive production methods without comprising the integrity of the organic semi-conductor and hence functionality of the OED. A wide-range of polymers may be employed provided they are soluble in a polar solvent and insoluble in water, capable of deposition on the organic semiconductor and have reactive moieties adapted to bind to a biomolecule. Preferably, the electronic properties of the OTFT prior to deposition of the capture layer and after its deposition are substantially the same.

The organic electronic device with its capture layer suitably provides a layered structure comprising an organic semi-conductor and a capture layer.

In another aspect, the invention provides a layered structure for binding to a biomolecule comprising an organic semi-conducting layer and a capture layer carried on the semi-conducting layer, the capture layer comprising a polymer which is substantially insoluble in water, the capture layer having a sample surface comprising moieties adapted to bind to the said biomolecule thereby to enable detection of a target species in use.

The capture layer polymer is soluble in a polar solvent and in which solvent the organic semi-conductor layer is insoluble. A solvent in which the polymers in the two adjacent layers are soluble and insoluble may be referred to as an orthogonal solvent in the present context. The capture layer polymer dissolved in the polar solvent may also suitably be formed into a layer by a deposition technique, for example by printing an ink composition and by spin-coating.

The organic semiconductor layer is preferably formed by a solution deposition method from a formulation comprising the or each component of the organic semiconductor layer dissolved or dispersed in a non-polar solvent, or a mixture of two or more non-polar solvents. At least one active component of the organic semiconductor layer is dissolved in the formulation. If more than one component is present, preferably each component is dissolved. Exemplary non-polar solvents are benzenes substituted with one or more substituents selected from C₁₋₁₀ alkyl and C₁₋₁₀ alkoxy groups, for example toluene, xylenes and methylanisoles. Non-polar aprotic solvents as described herein are preferably aprotic solvents having a dielectric constant at 20° C. of less than 8.

The polar solvent is suitably an organic solvent. The solvent may be aprotic provided it is not apolar. Suitably, the solvent is protic, preferably an alcohol or a fluorinated alcohol. Examples of preferred alcohols include alcohols having from 1 to 6 carbon atoms, for example methanol, ethanol, 1-propanol, isopropanol, 1-butanol, isobutanol, tertiary butanol and octafluoro-1-pentanol (OFP). Examples of other suitable polar solvents which allow deposition of the polymer in the capture layer without adverse interaction with the organic semi-conductor include dimethyl formamide, dimethyl acetamide and dimethyl sulphoxide. The solution in which the capture polymer is dissolved may contain only one polar solvent or a mixture thereof.

In a preferred embodiment the capture layer polymer is insoluble in water, soluble in a polar solvent and may be soluble or insoluble in apolar solvents. Preferably the solvent has a dielectric constant from 18 to 50. Some solvents, for example acetone and methyl ethyl ketone, having a dielectric constant in this range may not be suitable as they may adversely affect the organic semi-conducting layer. The skilled person will be readily able to test whether a particular solvent is suitable for use with a given organic semi-conducting layer.

Suitably, the capture layer comprises:
i. a copolymer comprising a first repeating unit and a second repeating unit which comprises moieties adapted to bind to the biomolecule wherein the first and second repeating unit are different; or
ii. a blend of a first polymer and a second polymer which second polymer comprises moieties adapted to bind to the biomolecule

The first repeating unit may be selected from a wide range of known repeating units and preferably is selected from repeating units for forming one or more of polystyrene, polyacrylate, polymethacrylate, polymethyl methacrylate and polycarbonate.

The second repeating unit comprises a derivatised repeating unit having moieties adapted to bind to the biomolecule. The derivatised repeating unit may be selected from a wide range of known repeating units for forming a polystyrene derivative, a polyacrylate derivate, a polymethacrylate derivate, a polymethyl methacrylate derivative and a polycarbonate derivate.

Suitably, the moieties adapted to bind the biomolecule comprise reactive groups able to react with biomolecules for example DNA, RNA, PNA, aptamers, peptides, antibodies, carbohydrates and fragments or parts of any one or more of the foregoing biomolecules. Preferably the reactive moieties comprise amine groups or reactive carboxy groups and the second repeating unit is derivatised with amine groups or carboxyl groups. It is especially preferred that the reactive moieties are amine groups. The amine group may be protected or protonated, for example by providing a protonated amine with a trifluoroacetate counter ion or other known protections systems.

The derivatised groups suitably increase the hydrophilicity of the polymer rendering it more soluble in the polar solvent than the comparable polymer without such derivatised groups. The degree of derivatisation may be varied to adjust the solubility of the derivatised polymer. The derivatised groups are suitably pendant to the polymer backbone. The derivatised groups may be covalently bound directly to the polymer backbone In a preferred embodiment the polymer is derivatised with amine groups which are pendant from the polymer backbone and bound thereto via a spacer moiety covalently bound to the polymer backbone. The spacer moiety is preferably selected from an alkylene group, for example methylene, ethylene and propylene, and a polyoxyalkylene group for example polyethylene glycol having from 1 to 4 ethylene oxide groups.

Examples of suitable spacer moieties with a reactive group include —CH₂CH₂NH₂, —OCH₂CH₂OCH₂CH₂NH₂. Where the polymer comprises styrene monomeric units, the spacer and reactive group are suitably linked to the styrene unit at the para position on the aromatic ring of the styrene unit for example a derivatised styrene monomeric unit of formula —[CH₂—CH(C₆H₄-para-OCH₂CH₂OCH₂CH₂NH₂)]—.

Preferably, the amine moiety is a primary amine. Secondary amines may be employed but may be less desirable if intended for binding a biomolecule due to greater steric hindrance.

Where a copolymer is employed, the mole ratio of the first repeating unit to the second repeating unit is suitably at least 0.1:1 and preferably at least 1:1. The mole ratio of the first repeating unit to the second repeating unit is preferably less than 50:1, more preferably less than 20:1, especially less than 10:1.

The derivative in the second repeating unit may impart hydrophilic characteristics to the second repeating unit. The first and second repeating units are selected and their ratio is selected such that the copolymer is insoluble in water and soluble in a polar solvent and the level of the derivative is such that the hydrophilic nature imparted to the copolymer is not sufficient to render the copolymer soluble in water. This would mean that any subsequent procedure to attach a biomolecule to the capture layer which is typically carried out in an aqueous medium or buffer, would render the capture layer unstable.

If the proportion of the hydrophilic derivative is too low, the copolymer may be too hydrophobic and not soluble in solvents compatible with the organic semi-conducting layer which may impair performance of the sensor.

Selection of a candidate copolymer may be carried out by synthesising one or more candidate copolymers for example having different molar ratios of the first and second repeating units or by selecting different repeating units and mixing the copolymer with a candidate solvent and subjecting to a solubility test to determine whether the polymer has dissolved. Solubility of the polymer may be tested by adding 100 mg polymer (pre-dried in a vacuum oven at 50° C. for 5 hours) to 1 mL water, stir the mix at room temperature and atmospheric pressure for 1 hour, filter and dry the polymer in a vacuum oven at 50° C. for 5 hours and reweigh the polymer. The polymer is insoluble if the weight of the re-weighed polymer is 100 mg. A substantially insoluble polymer as described herein may have a solid polymer weight of greater than 98 wt % or greater than 99 wt % of the starting polymer.

The candidate copolymer is also suitably tested to determine whether its deposition on an OSC results in delamination or deterioration.

The copolymer should be stable in contact with water or an aqueous solution. The term “stable” means that the copolymer does not dissolve or delaminate when in contact with water for a certain period and that the film remains intact for a sufficient period of time to allow the biomolecule to be attached to the surface of the capture layer and the sensing operation carried out. Desirably, the electronic characteristics of the OTFT prior to deposition of the capture layer and after deposition of the capture layer are substantially the same indicating that the OSC layer has not degraded. The electronic characteristics which desirably remain substantially the same are the threshold voltage, drain current, mobility and on/off ratio.

To determine whether the selected copolymer has suitable stability, the copolymer is coated for example spin-coated onto a substrate for example glass to form a deposited layer, a droplet of water is placed on the layer at room temperature and after 1 hour, the surface is then dried and subjected to visual inspection under a microscope at a magnification of ×10 and preferably at a magnification of up to ×50.

In a preferred embodiment, the capture layer polymer comprises a methacrylate copolymer having methacrylate repeating units and amine substituted methacrylate repeating units. A polymethyl methacrylate polymer may be expected to be soluble in non-polar solvents due to the large proportion of hydrophobic moieties but the present inventors have found that polymethyl methacrylate copolymer having a proportion of amine moieties is surprisingly soluble in a polar solvent, especially a polar protic solvent for example an alcohol, but not in water. Preferably, the amine substituted methacrylate repeating units are present in the copolymer at a level of 5 to 20 mole % of the repeating units.

The capture layer is suitably very thin so as to allow binding or interactioin with the target species to generate an electrical signal. The capture layer suitably has a thickness of 1 to 300 nm, preferably at least 5 nm and more preferably at least 10 nm. The capture layer has a thickness which is suitably up to 200 nm, preferably up to 100 nm, more preferably up to 70 nm and especially up to 50 nm.

The reactive moieties in the capture layer may be functionalised to tailor the sensor to specific applications and provide particular sensitivity or tuning for example for particular antibodies, DNA sequences or other species as desired.

In one embodiment the capture layer comprises a bound biomolecule and provides a sample surface of the OED for interaction and suitably capture of a target species such that it may be detected due to a change in the electronic signal in the sensor. The biomolecule is suitably immobilised and may be tailored for example to provide a DNA sequence complementary to a DNA sequence in a sample to be analysed or to provide an antibody to bind an antigen in a sample to be analysed which may be diagnostic of a particular condition.

In one embodiment, the sensor is a bioassay for detecting a DNA sequence in a DNA strand, a hormone or an antigen indicative of a disorder, for example a cancer marker. In a preferred embodiment, the biomolecule comprises DNA, RNA or a protein and the moieties in the capture layer comprise amine groups. Preferably, the sensor comprises a DNA probe or an antibody bound to the capture layer directly or indirectly.

The specificity of a low cost electronic biosensor, for example a printed OTFT, requires a stable layer of biomolecules to be bound to the capture layer directly or indirectly. The biomolecules selectively bind with a target species or analyte which provides an electronic signal. In a preferred embodiment, an OTFT sensor according to the invention comprises a bottom gate, low voltage OTFT comprising a bottom gate having a surface comprising bound a biomolecule.

Suitably, the biomolecule is bound via the moieties in the capture layer polymer or may be bound by unreacted terminal repeat units in the polymer. The moieties adapted to bind to the biomolecule are suitably bound to the biomolecule, preferably by a bioconjugation reaction.

The biomolecule may be any suitable biomolecule for example DNA, PNA, RNA, aptamers, peptides, antibodies, carbohydrates and parts or fragments of any of the foregoing biomolecules. In preferred examples the biomolecule comprises DNA, for example a DNA probe, or an antibody.

If desired, a label may also be bound directly or indirectly to the capture layer. The label may be a fluorescent label or an absorption label for example a latex or gold particle. However, binding of the target species provides an electronic signal and suitably is not dependent on the use of labels or markers in the target species.

In one embodiment, the moieties adapted to bind the biomolecule are adapted to bind to the biomolecule via a linker species. Preferably, the linker species comprises a homobifunctional cross-linker or a heterobifunctional cross-linker. Suitable cross-linkers include protein cross-linkers for example N-(6-maleimidocaproxy)succinimide (EMCS) and sulfo-ECMS. Attachment of DNA using ECMS linker is described in a published paper by Shimomura, A., Nishino, T., Maruyama, T. Langmuir 2013, 29, 932-938.

OTFTs are known in which the surface of the OTFT is coated with a blend of gold nanoparticles within a polymer matrix. Biomolecules may be immobilised on the nanoparticle surface through thiol-gold linkages. This approach however is limited to use with biomolecules having thiol groups and, furthermore, conjugates of biomolecules having thiol groups and gold may dissociate readily unless stored under carefully controlled optimal conditions.

In a preferred embodiment, the polymer in the capture layer in the present invention allows the biomolecule to be conjugated directly to the capture layer polymer. Advantageously, the sensor may be used with a wide range of biomolecules including those which do not have thiol groups.

Sensors, OEDs and layered structures according to the invention are suitably produced by depositing or printing the capture layer on the organic semi-conductor from a solution.

In a further aspect, a method is provided of making a layered structure for binding a biomolecule comprising:
i. providing a layer comprising an organic semi-conductor;
ii. providing a solution comprising a polar, non-aqueous solvent and a polymer which is soluble in the solvent and substantially insoluble in water and which polymer comprises moieties adapted to bind to the biomolecule; and
iii. depositing from the polymer from the solution onto the first layer to form a capture layer whereby the moieties are available to capture the biomolecule.

The polymer is suitably deposited onto the first layer by a printing process or a coating process. Known deposition processes may be employed, for example ink jet, lithographic, gravure and spin-coating. In a preferred embodiment, the polymer is deposited by spin-coating. In a preferred embodiment, spin coating is carried out using an air exposed spinner that uses a GYROSET™ lid, for example a Suss MicroTec Gamma System spin coater available from Suss MictroTec. The capture layer polymer solution is typically prepared using a solvent orthogonal to the OSC and any other materials of the organic semiconducting layer, if present, for example octafluoropentan-1-ol.

The or each material of the organic semiconductor layer is preferably substantially insoluble in the solvent or solvent mixture used to deposit the capture layer.

Solubility of the material or materials of the organic semiconducting layer in the solvent or solvent mixture used to deposit the capture layer may be tested by adding 100 mg of the material or material composition of the organic semiconducting layer (pre-dried in a vacuum oven at 50° C. for 5 hours) to 1mL of the solvent or solvent mixture, stir the mix at room temperature and atmospheric pressure for 1 hour, filter and dry the solid in a vacuum oven at 50° C. for 5 hours and reweigh the polymer. The material or materials are insoluble if the weight of the re-weighed solid is 100 mg. A substantially insoluble material or composition as described herein may have a solid weight upon re-weighing of greater than 98 wt % or greater than 99 wt % of the starting material or composition.

The film thickness may be varied by varying the amount of the polymer in the solvent. In one example, the solution comprises 1.3% of a polymethyl methacrylate copolymer in which 10% of the monomeric units have been derivatised with aminoethylene in octafluoropentan-1-ol and is spun at 1200rpm with an acceleration of 1000rpm for 30 seconds with a closed lid. The deposited wet film suitably has a thickness of about 100 nm.

Suitably, the solution is left standing on the OSC after it was flooded for 2 minutes to facilitate film wetting and formation. Following the spin coating, the substrate is removed from the spinner and transferred to a flat hot plate as rapidly as possible to reduce the risk of reticulation of the film on the edges of the substrate at a temperature of 80° C. and heated for 1 minute.

In another aspect, the invention provides a composition comprising a solution of a copolymer or polymer blend for coating on a surface to form a layer of the copolymer or polymer blend, the solution comprising:
A) i) a copolymer comprising a first repeating unit and a second repeating unit which comprises moieties adapted to bind to the biomolecule and in which the first and second repeating units are different; or ii) a blend of a first polymer and a second polymer which second polymer comprises moieties adapted to bind to the biomolecule; and
B) a polar solvent in which component A) is soluble.

The polar solvent is suitably as described herein. Suitably, the solvent is protic, preferably an alcohol or a fluorinated alcohol. Examples of preferred alcohols include alcohols having from 1 to 6 carbon atoms, for example methanol, ethanol, 1-propanol, isopropanol, 1-butanol, isobutanol, tertiary butanol and octafluoro-1-pentanol (OFP). Examples of other suitable polar solvents as described herein include dimethyl formamide, dimethyl acetamideand dimethyl sulphoxide.

FIG. 1 shows a sensor according to the invention having an electrical detection system and an optical detection system. The sensor has a low voltage transistor having a reflective gate. The organic light-emitting diode (OLED) and organic photodiode (OPD) enable electronic and optical detection of the bound labelled species in the same area The transistor gate may be reflective thereby allowing an absorption mode to be used. Alternatively, as desired, a colour filter may be used on the OPD enabling a fluorescence mode to be employed and avoiding the need to use a reflective gate electrode.

The invention is illustrated by the following non-limiting examples.

### EXAMPLE 1

A copolymer was synthesised from methyl methacrylate monomer as the first repeating unit and amine derivatised methyl methacrylate monomer as the second repeating unit in quantities such as to produce a copolymer in which 10 mole % of the repeating units contained an amine moiety.

Synthesis of Boc-Aminoethyl Methacrylate Monomer

A solution of diisopropylethyl amine (˜42 mL, 242 mmol) and N-Boc-ethanolamine (˜26 g, 161.3 mmol) in dry dichloromethane (500 mL) was cooled in an ice/water bath under nitrogen with stirring. A solution of methacryloyl chloride (˜18.5 g, 177.4 mmol) in dry dichloromethane (50 mL) was added at such a rate as to keep the internal temperature at ˜2□C. Once addition was complete, the colourless homogeneous solution was allowed to warm to ambient temperature overnight. The resulting homogeneous red solution was re-cooled in an ice/water bath and quenched by the addition of water dropwise keeping the internal temperature below 10° C. After warming to ˜15° C., the red solution was washed sequentially with citric acid solution (2×100 mL, ˜62 g in 200 mL water), potassium carbonate solution (2×100 mL, ˜45 g in 200 mL water), sodium hydrogen carbonate solution (2×100 mL, ˜27 g in 200 mL water) and brine (2×100 mL, 20 g in 200 mL water) with additional NaCl being added at the end to clear the emulsion that formed. The recovered organic layer was dried over anhydrous magnesium sulphate, filtered under gravity and concentrated to dryness to give a mobile dark red oil. The crude product was dissolved in heptane (200 mL), heated to 60° C. and filtered under gravity. After standing overnight, the resulting crystalline deposit was recovered by suction filtration and dried under suction in air to give the title compound as a pale orange crystalline solid (19.3 g, 52%)

Polymer Synthesis

A solution of methyl methacrylate (2.00 g, 20.0 mmol), boc-aminoethyl methacrylate (510 mg, 1.70 mmol) and 2,2′-azobis(2-methylpropionitrile) (10 mg, 0.061 mmol) in propylene glycol methyl ether acetate (3.76 g) was degassed and then stirred at 60° C. for 14 h under argon. After cooling to room temperature, the polymeric product was precipitated into an excess of propan-2-ol, collected by filtration and washed with further propanol-2-ol and dried in vacuo. To deprotect the pendant boc-groups, 5 g of polymer were dissolved in 100 mL of trifluoroacetic acid and this reaction mixture was stirred for 1 hour at room temperature. To precipitate the polymeric product, the reaction mixture was added dropwise to water (1 L) and, after collecting by filtration, washed with 5×200 mL of water and dried in vacuo.

The solubility of the copolymer was assessed in a range of solvents. The solvents were selected according to their relative orthogonality to the OSC layer, that is, the OSC layer was insoluble in the solvent. Dimethyl sulfoxide (DMSO) and octafluoropentan-1-ol (OFP) were tested.

A solution of the copolymer in OFP at a level of 1 w % by volume was spin-coated onto a glass surface onto an organic semi-conductor film on glass also from the same OFP solution and onto an organic semi-conductor film of Organic Semiconductor 1, illustrated below, on n-octadecyltrichlorosilane self-assembled monolayer (OTS-SAM) modified glass. The solution was left on the surface for 2 minutes before being spun. On deposition, the polymer formed a capture layer approximately 40 nm thick. The organic semiconductor film was formed by spin-coating a 1 wt % solution of Organic Semiconductor 1 in mixed xylenes.

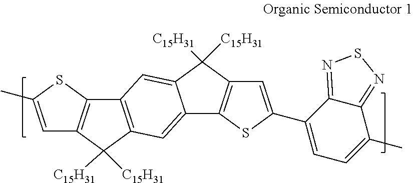

The capture polymer was also deposited by spin-coating onto the surface of a low voltage bottom-gate OTFT comprising an organic semiconducting layer formed by spin-coating a 1 wt % solution of Organic Semiconductor 1 in mixed xylenes. The capture polymer was deposited with a solution made in OFP at 1 w by volume % from a solution of the polymer in OFP. On deposition, the polymer formed a capture layer approximately 40 nm thick without deterioration or delamination of the organic semi-conductor layer. The transfer characteristics of the OTFT were tested before and after deposition of the capture layer. The transfer characteristics, namely threshold voltage, drain current, mobility and on/off ratio, were very similar before and after deposition demonstrating that the organic semi-conductor layer had not deteriorated or been delaminated or otherwise compromised in applying the capture layer.

### EXAMPLE 2

A 19 base DNA probe having a terminal thiol group (thiol-5′-DNA) was covalently attached to the capture layer of the OTFT product in Example 1 using a heterobifunctional cross-linker molecule, N-(6-maleimidocaproxy)succinimide (EMCS) according to the method described by Shimomura, A., Nishino, T., Maruyama, T. Langmuir 2013, 29, 932-938. The DNA probe was bound to a complementary fluorescent labelled DNA strand, FITC-5′-DNA, where FITC denotes fluorescein isothiocyanate, to provide a qualitative assessment of the bioconjugation steps. Detection of the labelled DNA demonstrated that the DNA probe had been successfully immobilised on the capture layer of the sensor.

### EXAMPLE 3

A copolymer of methyl methacrylate units and a derivatised styrene monomeric unit was prepared. The Styrene monomeric unit was N-(tert-butoxymethyl)-2-(2-(4-vinylphenoxy)ethoxy)ethan-1-amine, compound 8 in the synthetic scheme shown below.

### Synthesis of N-(tert-butoxymethyl)-2-(2-(4-vinylphenoxy)ethoxy)ethan-1-amine (8)

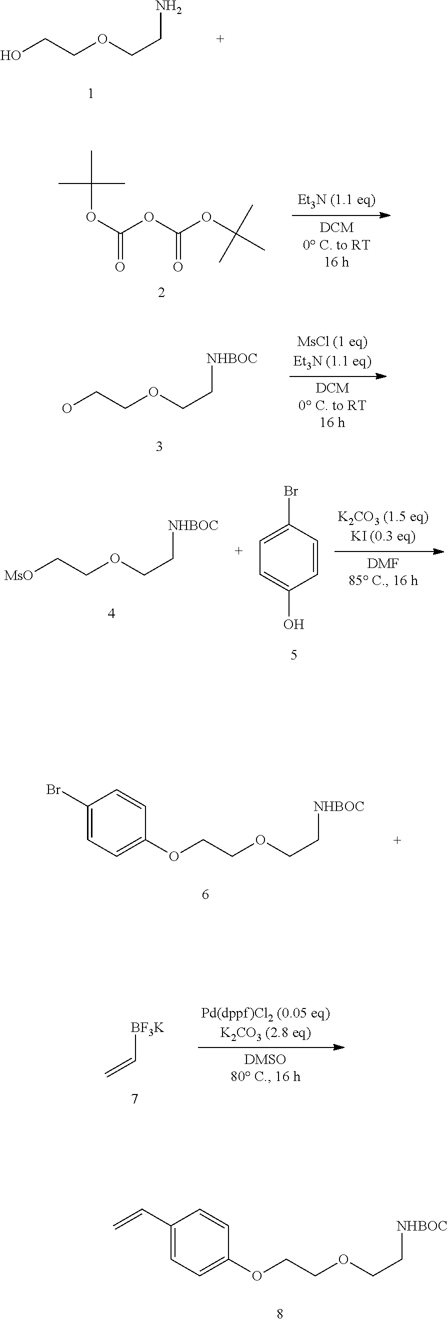

A solution of 2-(2-aminoethoxy)ethanol (30.0 g, 283 mmol) in dichloromethane (300 mL) was cooled to 0° C., followed by dropwise addition of trimethylamine (31.4 g, 311 mmol). After stirring at 0° C. for a further 20 min, di-tert-butyl dicarbonate (61.6 g, 283 mmol) was added.

The reaction mixture was allowed to heat to room temperature and was stirred at this temperature for 16 hours. Removal of the solvent in vacuo afforded intermediate 3 (58.0 g) which was used in the next step without further purification.

A solution of 3 (58.0 g, 282 mmol) in dichloromethane (300 mL) was cooled to 0° C., followed by dropwise addition of trimethylamine (31.4 g, 311 mmol). After stirring at 0° C. for a further 30 min, methanesulfonyl chloride (32.2 g, 282 mmol) was added. The reaction mixture was allowed to heat to room temperature and was stirred at this temperature for 16 hours. After this time, the reaction mixture was concentrated under reduced pressure and was diluted with ethyl acetate (750 mL). The organic phase was then washed with water (500 mL) and dried over magnesium sulfate. Removal of the solvent in vacuo afforded intermediate 4 (130 g) which was used in the next step without further purification.

To a solution of bromophenol (5) (60 g, 348 mmol) in dimethyl formamide (600 mL) was added potassium carbonate (72.2 g, 523 mmol), followed by potassium carbonate (17.3 g, 104 mmol) and 4 (128 g, 45.3 mmol). The reaction mixture was then heated to 85° C. and was heated at this temperature for 16 hours. After this time the reaction mixture was cooled to room temperature and was concentrated under reduced pressure, followed by addition of water (1 L). The aqueous phase was extracted with ethyl acetate (2×1 L) and the combined organic phases were dried over sodium sulfate. Removal the solvent in vacuo and purification of the crude product by flash silica column chromatography (eluting with ethyl acetate/hexane) afforded intermediate 6 (85.0 g, 236 mmol, 83% over 3 steps).

To a solution of intermediate 6 (25.0 g, 69.0 mmol) in dimethylsulfoxide (250 mL) was added trifluoro vinyl borate (7) (27.7 g, 208 mmol) and potassium carbonate (26.8 g, 194 mmol) and the suspension was degassed by bubbling with nitrogen for 30 minutes. After this time, Pd(dppf)Cl2 (2.83 g, 3.00 mmol) was added and the solution was degassed for a further 30 minutes. The reaction mixture was then heated to 80° C. and was allowed to stir at this temperature for 16 hours. After cooling to room temperature, the reaction mixture was filtered and diluted with ethyl actetate (500 mL) before washing with water (200 mL) and brine (200 mL). The organic phase was dried over sodium sulfate and concentrated under reduced pressure. The crude product was purified by flash silica column chromatography (eluting with ethyl acetate and hexane) and further triturated with hexane (100 mL) for 5 hours to afford monomer 8 (5.3 g, 17.2 mmol, 58%).

### Synthesis of co-polymer of MMA and monomer 8 and boc-deprotection

The polymer synthesis method described above in Example 1, was used for the co-polymerisation of methyl methacrylate (3.00 g, 3.00 mmol) and monomer 8 (1.02 g, 33.2 mmol). After isolation of the polymeric product, pendant boc-groups were deprotected by treatment with trifluoroacetic acid, as described above in Example 1.

The solubility of the polymer was tested in a range of solvents by adding 100 mg polymer (pre-dried in a vacuum oven at 50° C. for 5 hours) to 1mL water, stirring the mix at room temperature for 1 hour, filtering and drying the polymer in a vacuum oven at 50° C. for 5 hours and reweighing the polymer. The polymer was found to be insoluble in water (solid polymer weight before and after stirring with water was unchanged) and soluble in dimethyl sulfoxide (DMSO) and octafluoropentan-1-ol (OFP).

1. A method of making a layered structure for binding a biomolecule comprising:
forming an organic semi-conductor layer by a solution deposition method;
forming a capture layer on and in contact with the organic semiconductor layer comprising the step of depositing a solution comprising a polar, non-aqueous solvent and a dissolved capture polymer onto the organic semi-conductor layer wherein the capture polymer comprises moieties adapted to bind to a biomolecule.

2. A method according to claim 1 wherein the capture polymer is substantially insoluble in water.

3. A method according to claim 1 in which the capture polymer is deposited onto the first layer by a printing process or a coating process.

4. A method according to claim 1 wherein the polar nonaqueous solvent has a dielectric constant from 18 to 50.

5. A method according to claim 1 wherein the polar non-aqueous solvent is a protic solvent.

6. A method according to claim 1 wherein the organic semiconductor layer is formed from a formulation comprising a non-polar solvent and an organic semiconductor.

7. A method according to claim 6 wherein the non-polar solvent has a dielectric constant of less than 8.

8. A method according to claim 1 wherein the moieties are selected from amine groups; carboxyl groups; and salts thereof.

9. A method according to claim 1 wherein the capture polymer is a copolymer comprising repeat units that are substituted with at least one moiety adapted to bind to a biomolecule and repeat units that are not substituted with a moiety adapted to bind the biomolecule.

10. A method according to claim 1 wherein the capture polymer comprises repeat units selected from one or more styrene, acrylate, methacrylate, methyl methacrylate and carbonate repeat units.

11. A method according to claim 1 wherein the organic semiconductor layer is a layer of an organic thin-film transistor.

12. A layered structure obtainable by a method according to claim 1.

13. A method of forming a sensor comprising the step of binding a biomolecule to the moieties of the layer structure according to claim 12.

14-44. (canceled)

45. A method of making a layered structure for binding a biomolecule comprising:
i) providing a first layer comprising an organic semi-conductor;
ii) providing a solution comprising a polar, non-aqueous solvent and a polymer which is soluble in the solvent and substantially insoluble in water and which polymer comprises moieties adapted to bind to the biomolecule; and
iii) depositing from the polymer from the solution onto the first layer to form a capture layer whereby the moieties are available to capture the biomolecule.

46. A method according to claim 45 in which the polymer is deposited onto the first layer by a printing process or a coating process.

47. A method according to claim 46 in which the deposition comprises spin-coating.

48-55. (canceled)
